(19) United States
(12) Patent Application Publication
(10) Pub. No.: US 2002/0119143 A1
(43) Pub. Date: Aug. 29, 2002

(54) Antithrombin III for disorders caused by angiogenesis

(76) Inventors: C. Jane Robinson, Hertfordshire (GB); Elaine Gray, London (GB); Christian Josef Wiedermann, Innsbruck (AT); Juergen Roemisch, Marburg (DE); Harald Stauss, Dautphetal (DE)

Correspondence Address: Finnegan, Henderson, Farabow,, Garrett & Dunner, L.L.P., 1300 I Street, N.W., Washington, DC, 20005-3315, US

(21) Appl. No.: 10/046,278

(22) Filed: Jan. 16, 2002

(30) Foreign Application Priority Data
Jan. 17, 2001 (DE) 101 02 048.1

(51) Int. Cl. A61K 38/48

(52) U.S. Cl. 424/94.63

(57) ABSTRACT

The use of active antithrombin III which has thrombin-inhibitory properties and affinity for heparin for the prophylaxis and treatment of disorders caused by pathological angiogenesis or arteriogenesis, is described.

The invention relates to the use of the antiangiogenic and antiarteriogenic activity of antithrombin III for the prophylaxis and treatment of various disorders.

Angiogenesis means the growth of capillary vessels and the growth of endothelial channels, whereas arteriogenesis refers to the growth of collateral vessels which are already present, together with the extension of the arteries which are present and are provided with muscles (1). Both processes are initiated by the binding of substances with angiogenic activity to receptors which are located on endothelial cells which then proliferate and migrate away. In parallel with this, stimulated endothelial cells also increase the formation of adhesion molecules (integrins) such as αᵥβ₃, which serve to anchor the endothelial cells which have migrated away to the surrounding tissue, leading to a sprouting of new blood vessels. In addition, there is formation of metalloproteinases which break down the surrounding tissue and thus make it possible for the tissue to form anew around the blood vessels. The sprouting endothelial cells penetrate into tubular and loop-shaped recesses and thus make the formation of blood vessels possible. Since angiogenic agents play a crucial part in angiogenesis and arteriogenesis, an enhancement or reduction in their production and effects has a large influence on the normal physiological control of these processes and on disorders influenced by angiogenesis. Pathological angiogenesis is characteristic of cancer and various ischemic and inflammatory disorders. There is evidence of the important part played by substances with angiogenic activity and growth factors in the growth and formation of metastases of cancer cells (2). It is certain that excessive angiogenesis may lead to disorders such as diabetic retinopathy, neuropathy, rheumatoid arthritis, psoriasis and endometriosis. Angiogenesis contributes to pathophysiological tissue changes associated with chronic bronchitis and chronic inflammations of the gastrointestinal tract and to granulomatous and other infectious diseases such as leprosy.

Antithrombin is one of the principal endogenous inhibitors of coagulation. Although antithrombin acts in particular as an important thrombin inhibitor in the plasma, it also has strong inhibitory effects on a number of active serine proteases including factors IXa, Xa, XIa and XIIa and on factor VIIa bound to tissue factor, all of which are important for the coagulation cascade. Two isoforms of antithrombin have been identified in human plasma. The β isoform accounts for 5 to 10% of plasma antithrombin and has a greater heparin affinity than the α isoform. However, the proportions of these two isoforms vary with the tissue from which they are isolated (3) and, depending on the isolation method used, different antithrombin concentrates also contain different amounts of the isoforms (4).

Recently, O'Reilly et al. (5) described the antiangiogenic and antitumor activity of the cleaved and latent forms of antithrombin, while the active antithrombin (AT) did not show such properties. O'Reilly et al. found, by fractionating the cell culture supernatant, a new antiangiogenic protein which was identified as antithrombin and in which the so-called active loop was cleaved, which led to loss of its inhibitory properties in relation to the known proteases such as thrombin. This proteolytic cut was accomplished by elastase. The change in the conformation of AT after isolation can be brought about in a similar way by heat treatment and then results in the so-called locked or latent AT.

It has now been found, surprisingly, that the active form of AT, which is defined by intact molecules with the ability to inhibit proteases such as thrombin and factor XIa, and by a strong interaction with heparin and related compounds, has both antiangiogenic and antiarteriogenic properties. It is therefore possible to employ the active form of AT as medicament for the prophylaxis and treatment of disorders arising through pathological angiogenesis and arteriogenesis.

In a series of experiments, firstly the inhibitory effects of the active forms of antithrombin, including the α and β forms of antithrombin, on endothelial cell proliferation induced by growth factors were investigated. The effects of these active isoforms on the serum-induced proliferation of human umbilical vein endothelial cells (HUVEC) and calf pulmonary arterial cells (CPAC) were then investigated. AT α and β were prepared by fractionated chromatography using a heparin matrix. Under these conditions, the latent antithrombin appeared in the fraction flowing through the column, while the α isoform was obtained by elution with 0.8 M NaCl and the β isoform was then obtained by elution with 2 M NaCl. By use of so-called two-dimensional immunoelectrophoresis (in the presence of heparin), the absence of the latent/locked AT in the two latter fractions was confirmed. In addition, the resulting AT shows full protease-inhibitory properties.

It can thus be stated, in summary, that both active AT isoforms show antiproliferative properties on incubation with endothelial cells. The inhibitory strength shown by the β isoform was greater than that of the α isoform. An AT concentrate containing a mixture of both active isoforms likewise showed inhibitory activity. The presence of an amount (10%) of latent AT did not reduce the inhibitory strength of the concentrate.

The invention therefore also relates to the use of the α isoform or of the β isoform or of a mixture thereof or of a concentrate of antithrombin III for the prophylaxis and treatment of disorders caused by pathological angiogenesis or arteriogenesis.

It has also been possible to show that endothelial cell proliferation induced either by growth factors such as VEGF (vascular endothelial growth factor) or basic fibroblast growth factor (bFGF) or serum can be inhibited by active AT or an AT concentrate. The use of an active AT preparation prepared by immunoadsorption showed comparable results and confirmed that the angiogenic activity is mediated by AT and not, for example, by traces of other plasma proteins. It can be concluded from this that active AT, specifically either the active α or β isoforms, alone or as mixture, can be used for the prophylaxis and treatment of disorders induced by angiogenesis or assisted by it or accompanied by it, such as retinopathies, neuropathies, rheumatoid arthritis, psoriasis, endometriosis, and that they can also be used to prevent the spread of metastases and the growth of tumors, including those induced or assisted by growth factors such as cytokines. The same applies to the prophylaxis and treatment of chronic bronchitis and chronic inflammations of the gastrointestinal tract and granulomatous and other infectious diseases such as leprosy. The presence of latent AT does not reduce the antiangiogenic properties which have been found, so that a mixture containing active α- and/or β-AT can likewise be used. Apart from antithrombin obtained from plasma, it is also possible to use active antithrombin prepared recombinantly or transgenically, in particular either alone or in combination with latent antithrombin.

Antithrombin can be employed intravenously, subcutaneously, intramuscularly or topically (for example in the form of drops, ointments or as component of a means for wound closure, such as a fabric). The following examples show the inhibitory effects observed with the purified AT isoforms and an AT concentrate.

### EXAMPLE 1

Inhibition of VEGF-induced HUVEC Proliferation by Antithrombin

It was possible to show that VEGF₁₀₅ is able to induce a dose-dependent increase in the number of HUVEC cells, which was measured by staining with crystal violet. Incubation with 15.6 ng/ml VEGF (a concentration which produces a submaximal effect) was carried out in the presence of various concentrations of different preparations and fractions of antithrombin in RPMI 1640 for 48 hours.

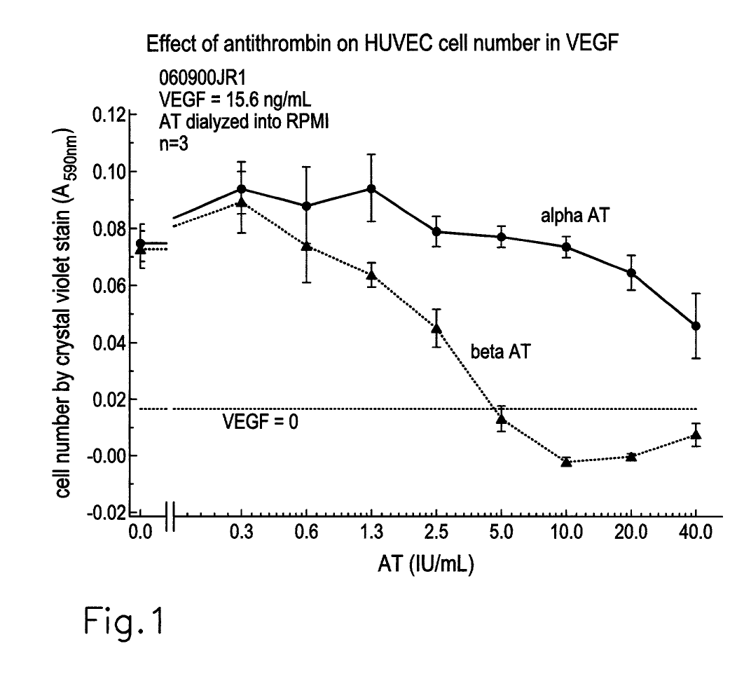

The effect of AT was a dose-dependent inhibition of the VEGF-induced increase in the number of HUVEC. The β isoform was more effective than AT-α, as shown by FIG. 1.

### EXAMPLE 2

Inhibition of Endothelial Cell Proliferation by an AT Concentrate

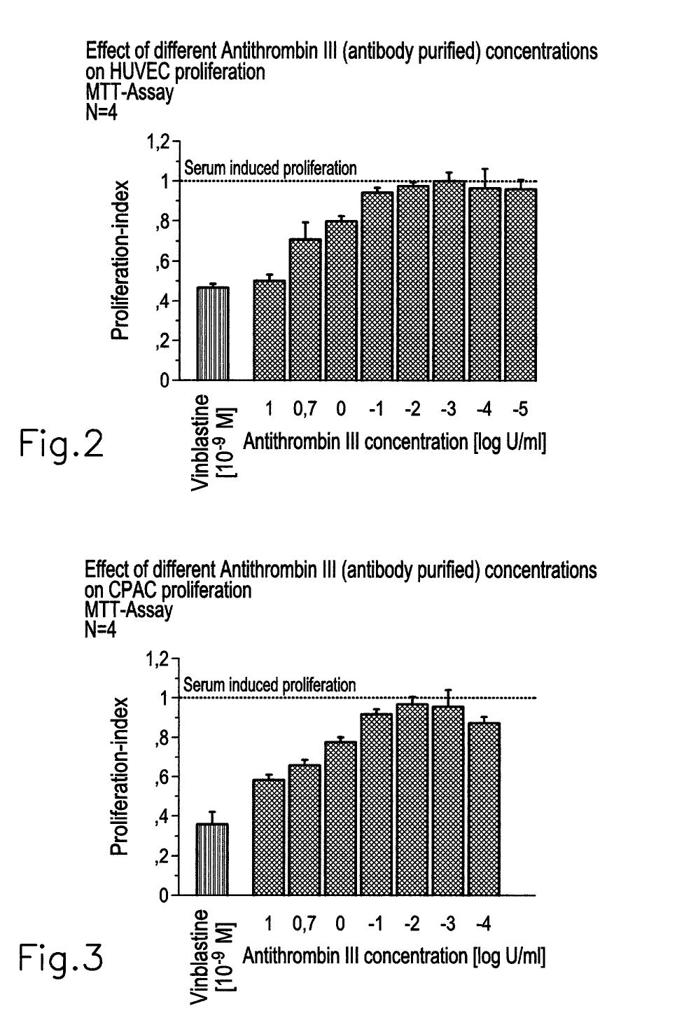

HUVEC was isolated from fresh placental umbilical cords and allowed to grow to confluence in a moist atmosphere with 5% CO₂ at 37° C. The growth medium was ECGM (PromoCell, Heidelberg, Germany) supplemented with 10% fetal calf serum (FCS) (PAA Laboratories, Linz, Austria). The cells were then separated from one another by treatment with collagenase and seeded in a culture medium which contained 20% FCS in a concentration of 5×10³ cells per well of a tissue culture plate equipped with 96 wells. After 24 hours, the cells were washed twice with RPMI 1640 (Biological Industries, Kibbutz Beit Haemek, Israel) and incubated with the test substances in a medium containing 2% FCS for 72 hours. Vinblastine was employed in a concentration of 10⁻⁹ M as positive control (see FIG. 2). The antiproliferative effect of this substance on HUVEC has already been described (6). A second endothelial cell line, the bovine pulmonary artery endothelial cell line CPA (ATCC, Rockville, Md.) was used together with a culture medium which consisted of Earle's Medium 199 (PAA Laboratories, Linz, Austria). The amounts of FCS were as described above (see FIG. 3).

After incubation at 37° C. for the stated time, the cell proliferation was measured using a colorimetric assay system. This assay system is based on the reaction of the tetrazolium salt MTT (Sigma Chemical Company) to give a violet formazan through active mitochondrial dehydrogenase. This reaction thus indicates live but not dead cells, and the signal generated is directly proportional to the number of cells. The MTT solution was added at a concentration of 5 mg MTT/ml PBS to all the wells of the assay culture plate and incubated for a further 6 hours. Then DMSO (Merck) was added to each well, and the plates were incubated for a further 30 minutes. The optical density was then measured in an enzyme-linked immunosorbent assay (ELISA) Reader at 570 nm.

In order to confirm these results, a BrdU assay system (Boehringer Mannheim, Germany) was used in accordance with the manufacturer's instructions. This assay system is based on measuring the incorporation of BrdU during DNA synthesis in proliferating cells.

The data are indicated as proliferation index which indicates the ratio between the serum-induced cell proliferation and the cell proliferation in the presence of test substances.

### EXAMPLE 3

Effect of an AT Concentrate on the Proliferation of HUTVEC and CPA

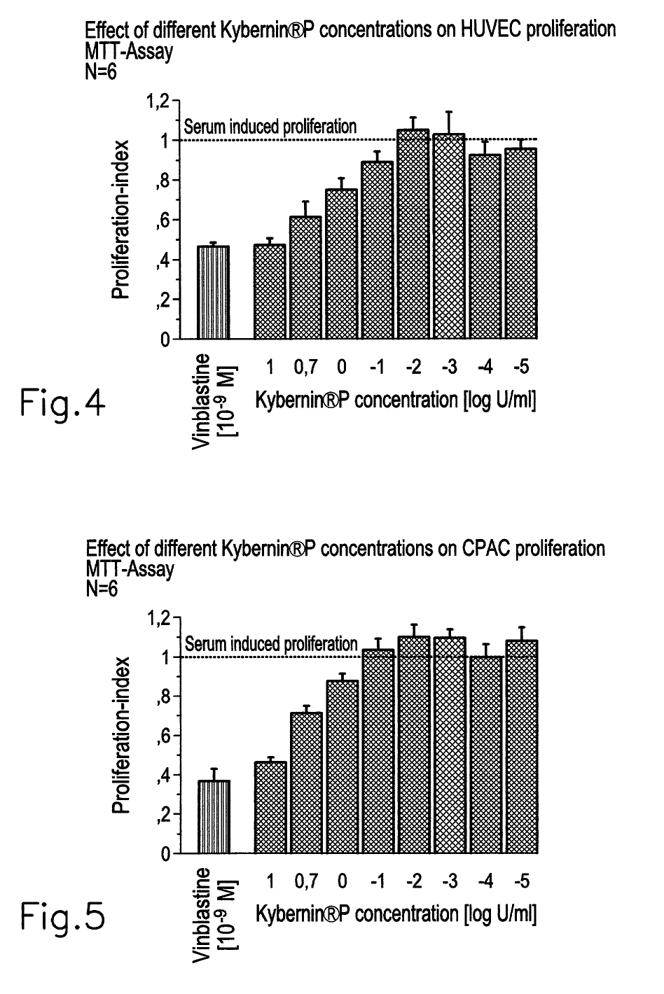

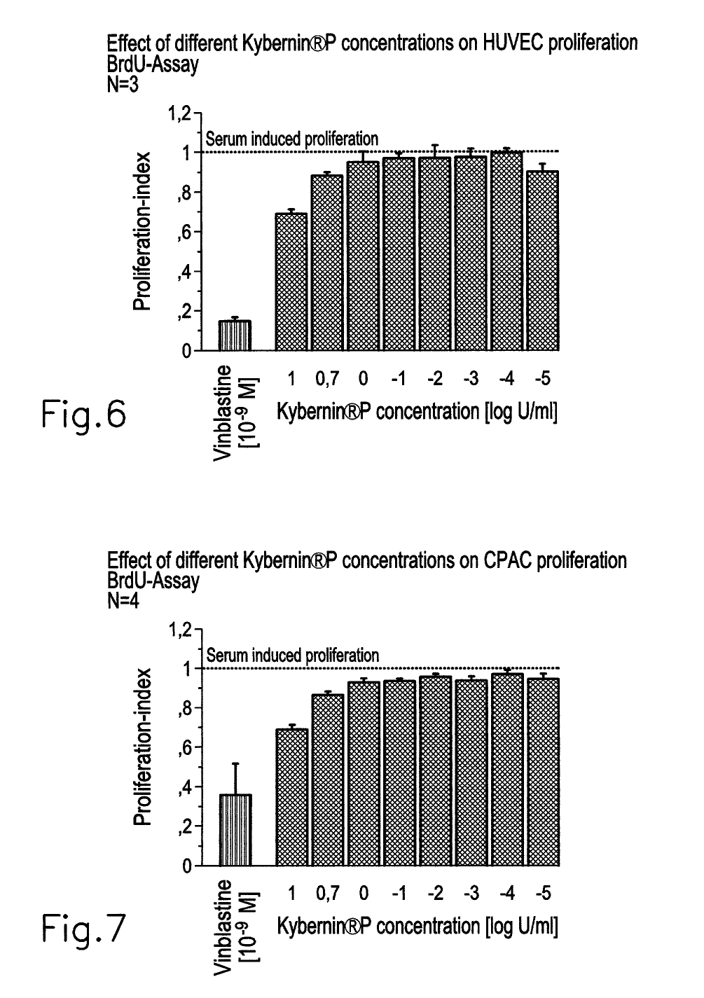

An AT concentrate (Kybernin ®P, Aventis Behring GmbH, Germany) which contained about 10% latent AT inhibited the proliferation of HUVEC or CPA cells in a concentration-dependent manner (above 1 IU/ml) when it was added to the culture medium before starting the 72-hour incubation. This observation shows that the mixture of active (in relation to protease inhibition and the binding to heparin) and latent AT likewise shows inhibitory properties on cell proliferation. In order to confirm that the reduced number of endothelial cells in the MTT assay (FIG. 4 and FIG. 5) actually is attributable to the inhibition of DNA proliferation, the synthesis was carried out in endothelial cells by means of a BrdU incorporation assay (FIG. 6 and FIG. 7). The results of the AT III inhibition on DNA synthesis with such concentrates show their antiproliferative effects.

A mixture of purified AT α and β (without latent AT) likewise showed an inhibitory effect in these assay systems.

### REFERENCES:

1. Carmeliet P. Mechanisms of angiogenesis and arteriogenesis. Nature Med. 2000; 6: 389-395.

2. Abdulkadir S A. Carvalhal G F. Kaleem Z., Kisiel W. Humphrey P A, Catalona W J, Milbrandt J. Tissue factor expression and angiogenesis in human prostate carcinoma. Hum Pathol. 2000; 31 (4): 443-447.

3. Witmer M R and Hatton M W. Antithrombin III beta associates more readily than antithrombin III-alpha with uninjured and de-endothelialized aortic wall in vitro and in vivo. Arterioscler Thromb. 1991; 11 (3): 530-539.

4. Römisch J., Dönges R., Stauss H., Inthorn D., Mühlbayer D., Hoffmann J N. AT III isoform proportion in plasmas of healthy subjects, septic patients and AT III concentrates a novel method of quantitation. Intens Care Med 2000: 26 (3):S 303, A 345.

5. O'Reilly M S, Pirie-Shepherd S., Lane W S and Folkman J. Antiangiogenic activity of the cleaved conformation of the serpin antithrombin. Science. 1999; 285 (5435): 1926-1928.

6. Vacca A., Iurlaro M., Ribatti D., Minischetti M., Nico B., Ria R., Pellegrino A., Dammacco F., Antiangiogenesis is produced by nontoxic doses of vinblastine. Blood. 1999: 94 (12): 4143-55.

Patent claims:

1. The use of active antithrombin III which has thrombin-inhibitory properties and affinity for heparin for the prophylaxis and treatment of disorders caused by angiogenesis or arteriogenesis.

2. The use as claimed in claim 1 of antithrombin III which contains active antithrombin for the prophylaxis and the treatment of disorders caused by angiogenesis or arteriogenesis.

3. The use of antithrombin III as claimed in claims 1 and 2, wherein the α isoform, the β isoform, mixtures of the two or a concentrate of antithrombin III are used for the prophylaxis and the treatment of disorders caused by angiogenesis or arteriogenesis.

4. The use of antithrombin III as claimed in claims 1 to 3, wherein it is employed for the prophylaxis and the treatment of retinopathies, neuropathies and infectious diseases such as leprosy.

5. The use of antithrombin as claimed in claims 1 to 3, wherein it is employed for the prophylaxis and treatment of cancerous ulcers and metastases of cancerous ulcers.

6. The use of antithrombin as claimed in claims 1 to 5, wherein it is administered intravenously, subcutaneously, intramuscularly or topically.
